(19) United States
(12) Patent Application Publication
(10) Pub. No.: US 2012/0029606 A1
(43) Pub. Date: Feb. 2, 2012

(54) Cooling device for use with heat-exchange catheter and method of use

(76) Inventors: Roy C. Leguidleguid, Union City, CA (US); Jeffrey Bleam, Boulder Creek, CA (US); Sam Omaleki, Morgan Hill, CA (US); Hoang Phan, Fremont, CA (US); George Tsai, Sunnyvale, CA (US)

(21) Appl. No.: 11/596,784

(22) PCT Filed: May 18, 2005
(86) PCT No.: PCT/US05/17475

§ 371 Date: Aug. 29, 2011

Related U.S. Application Data
(60) Provisional application No. 60/572,199, filed on May 18, 2004.

(51) Int. Cl. A61F 7/12

(52) U.S. Cl. 607/105

(57) ABSTRACT

Devices, methods and systems for endovascular cooling of human or animal subjects are disclosed. A heat exchange catheter (12) having a heat exchanger (22) is attached to a source of a thermal exchange fluid (32) that circulates through the heat exchanger, thereby cooling the subject's flowing blood. This invention provides cooling devices, systems and methods for cooling the thermal exchange fluid more rapidly and/or to a lower temperature than was achieved by the prior art.

### RELATED APPLICATION

This application claims priority to U.S. Provisional Patent Application No. 60/572,199 filed on May 18, 2004, the entirety of which is expressly incorporated herein by reference.

### FIELD OF THE INVENTION

This invention relates generally to medical devices, systems and methods and more particularly to devices, systems and methods for endovascular cooling of human or animal subjects.

### BACKGROUND OF THE INVENTION

This invention relates generally to medical devices for selectively controlling the temperature of a patient's body, or portion of the patient's body, by adding or removing heat from the patient's body fluid. More particularly, this invention relates to apparatus and method for reducing the temperature of a heat exchange fluid before the fluid is circulated through a heat-exchange catheter.

Heat exchange catheters are frequently used for raising or lowering the body temperature of patients suffering from either hyperthermia or hypothermia due to conditions such as exposure to a very hot or cold environment. Such catheters are also used to induce hypothermia in initially normothermic patients undergoing cardiovascular and/or neurosurgery and to victims of head trauma, spinal trauma, stroke, spinal surgery, aneurysm repair and the like, since hypothermia has a recognized neuroprotectant effect.

One type of heat exchange system available for medical use comprises a catheter having at least one heat transfer surface and at least one flow lumen which permits flow of a fluid past the heat transfer surface. The flow lumen receives fluid from an extracorporeal thermoelectric heat exchanger coupled to a control module including a pump or other discharge assistant and various controls for controlling the amount and direction of heat exchange occurring through the heat exchanger. Examples of prior art heat exchange catheter systems are disclosed in U.S. Pat. No. 5,486,208 to Ginsburg, U.S. Pat. No. 6,610,083 to Keller et al., U.S. Pat. No. 6,620,188 to Ginsburg, legally incapacitated, et al, and U.S. Pat. No. 6,673,098 to Machold et al. The complete disclosures of each of these patents are incorporated herein by reference.

Under certain circumstances, the thermoelectric heat exchangers used in the heat exchange catheter systems described above may not be able to cool the heat exchange fluid as quickly as required. Accordingly, there exists a need for cooling devices that can be incorporated into new or pre-existing heat exchange catheter systems, either in lieu of or in addition to a thermoelectric heat exchanger. Ideally, such devices should be free of moving mechanical parts, allowing the devices to be manufactured and maintained simply, quickly, and economically.

### SUMMARY OF THE INVENTION

The present invention provides a cooling device for use with a heat exchange catheter having at least one heat transfer surface and at least one flow lumen which permits flow of a fluid past the heat transfer surface. The cooling device includes an inlet configured to be connected in fluid communication with a fluid source upstream of the cooling device, a chamber containing a cooling medium, and an outlet configured to be connected in fluid communication with the flow lumen of the intravascular catheter to allow cooled fluid to pass from the chamber to the flow lumen. The cooling medium, which may be, for instance, a frozen liquid or gel, or a combination of ice and liquid, is preferably maintained in a substantially still condition in the chamber. Thus, no additional ducting or discharge assistants are required for circulating the cooling medium through the chamber.

In one embodiment, the cooling device comprises a substantially cylindrical or plate-shaped vessel having each end sealed with an end cap. The interior of the vessel between the two end caps is filled with a liquid, such as water, or a gel. An inlet tube extending through one of the end caps carries a first quick-disconnect fastener for detachably coupling the inlet tube in fluid communication with the fluid source, and an outlet tube extending through the other end cap carries a second quick-disconnect fastener for detachably coupling the outlet tube in fluid communication with the flow lumen of the heat exchange catheter. A tubular helical coil extends between the inlet tube and the outlet tube, allowing heat exchange fluid such as, for instance, saline solution, to flow past the liquid or gel in the chamber.

The cooling device according to this embodiment is configured for convenient storage in a cold environment such as a freezer, which maintains the cooling medium (i.e. the liquid or gel) in the vessel at a very cold temperature. The vessel may be stored at a temperature at or below the freezing point of the cooling medium so that the cooling medium is in a solid state when initially removed from storage. When needed, the device is simply removed from the freezer and detachably coupled between the fluid source and the inlet lumen of the heat exchange catheter. The fluid from the fluid source then circulates through the helical coil past the cooling medium, and enters the heat exchange catheter at a reduced temperature. If at some point the temperature of the cooling medium becomes too high to effectively cool the heat exchange fluid, the cooling device may quickly be removed from the system and replaced with a fresh cooling device from the freezer.

In an alternate embodiment, the cooling device is a direct contact heat exchanger in the form of a vessel, for instance an insulated pressure vessel, filled with a cooling medium such as an ice and saline bath. The vessel includes an inlet configured to be coupled to a fluid source and an outlet configured to be coupled to the inlet lumen of a heat exchange catheter, with the outlet positioned relative to the inlet so that flow from the inlet to the outlet is assisted by gravity. Advantageously, the inlet opens into an upper portion of the chamber and the outlet is in the form of an outlet tube having an entrance end opening into a lower portion of the chamber and an exit end proximate the upper end of the chamber. Both the inlet and the outlet tube may extend through a removable cap that is mounted in sealing engagement with the side wall of the vessel. The removable cap allows a user to easily add more ice or the like if the cooling medium becomes too warm.

A method of cooling a heat transfer fluid for use in a heat exchange catheter according to the present invention comprises the steps of providing a cooling medium in a vessel having an inlet and an outlet and defining an interior chamber, detachably coupling the inlet of the vessel in fluid communication with a fluid source containing the heat transfer fluid and the outlet of the vessel to the flow lumen of the heat exchange catheter, passing the heat transfer fluid through the interior chamber to allow heat exchange between the cooling medium and the heat transfer fluid, and passing the heat transfer fluid from the interior chamber to the flow lumen of the heat exchange catheter. The method optionally also includes steps of storing the vessel in a freezer when not in use and replacing the vessel with a similar, freshly frozen vessel when the temperature of the cooling medium in the vessel exceeds a predetermined level. Alternatively, the method may include a step of adding ice or the like to the vessel when the cooling medium becomes too warm.

The cooling device of the present invention may be easily incorporated to a pre-existing heat-exchange catheter system, either in lieu of or in addition to the thermoelectrically controlled cooler that it is conventionally provided with such systems. The heat exchange catheter system may also include a pump for assisting the flow of the heat transfer fluid from the fluid source to the inflow lumen of the heat exchange catheter.

Additional aspects and advantages of the present invention are set forth in the following description and claims, particularly when considered in conjunction with the accompanying drawings in which like parts bear like reference numerals.

### BRIEF DESCRIPTION OF THE DRAWINGS

FIG. 1 is a perspective view showing a prior art heat exchange catheter system;

FIG. 2 is a perspective view, partially broken away, of a cooling device according to one embodiment of the present invention;

FIG. 3 is a longitudinal sectional view through the cooling device of FIG. 2; and

FIG. 4 is longitudinal sectional view through a cooling device according to an alternate embodiment of the present invention.

### DETAILED DESCRIPTION OF THE EMBODIMENTS

FIG. 1 illustrates a prior art heat exchange catheter system 10 including a heat exchange catheter 12, a heat exchange cassette 14, and a control module 16 which houses a fluid source 16, for instance an IV bag of saline, and various controls such as a thermoelectric cooler (not shown) for adding or withdrawing heat from the heat exchange cassette 14. The system may also include a pump (not shown), which may either be part of or external to the heat exchange cassette 14, for circulating heat exchange fluid through the system.

The heat exchange catheter 12 is formed with an inlet flow line 18, an outlet flow line 20, and a heat exchange element 22 which may be, for instance, a heat exchange balloon. The inlet and outlet flow lines 18, 20 define inflow and outflow lumens (not shown), respectively, that are coupled to corresponding openings in the heat exchange cassette 14. The catheter 12 may, for instance, be any of the types disclosed in U.S. Pat. No. 5,486,208 to Ginsburg, U.S. Pat. No. 6,610,083 to Keller et al. and U.S. Pat. No. 6,620,188 to Ginsburg, legally incapacitated, et al., the entire contents of which are expressly incorporated herein by reference.

FIGS. 2 and 3 show one embodiment of a cooling device 24 that may be incorporated into the heat exchange catheter system 10 of the present invention, either in place of or in line with the thermoelectrically controlled heat exchange cassette 14. The illustrated cooling device 24 comprises a cylindrical vessel 26 having each end sealed with an end cap 28, 30. Although shown here as substantially cylindrical, the vessel 26 may also be structured as a flat plate or other suitable configuration.

The interior of the vessel 26 between the two end caps 28, 30 defines a chamber filled with a cooling medium 32, which may be a liquid, such as water, or a gel. An inlet tube 34 extending through one of the end caps 28 carries a first quick-disconnect fastener 36 for detachably coupling the inlet tube 34 in fluid communication with a fluid source such as an IV bag of saline, and an outlet tube 38 extending through the other end cap 30 carries a second quick-disconnect fastener 40 for detachably coupling the outlet tube 38 in fluid communication with a flow lumen of a heat exchange catheter. A tubular helical coil 42 extends between the inlet tube 34 and the outlet tube 38, allowing heat exchange fluid such as, for instance, saline solution, to flow past the cooling medium 32 in the vessel 26. The helical coil 42 may be made of any suitable thermally conductive material such as metal or plastic in order to allow rapid heat exchange between the heat exchange fluid and the cooling medium 32, while the vessel 26 may be insulated to prevent heat exchange between the cooling medium 32 and the surrounding air.

The cooling medium 32 is preferably substantially still. In other words, the cooling medium need not circulate. This makes the cooling device 24 substantially simpler, less expensive, and easier to maintain than, for instance a parallel, counterflow, or cross-flow type heat exchanger which would require additional ducting and/or moving mechanical parts to keep the cooling medium flowing.

A method of cooling a heat transfer fluid for use in a heat exchange catheter according to the present invention comprises detachably coupling the inlet tube 34 of the device 24 to a fluid source containing the heat transfer fluid and the outlet tube 38 to the flow lumen of the heat exchange catheter, passing the heat transfer fluid through the interior chamber of the vessel 26 to allow heat exchange between the cooling medium 32 and the heat transfer fluid, and passing the heat transfer fluid from the interior chamber 32 to the flow lumen of the heat exchange catheter. The vessel 26 containing the cooling medium 32 is preferably stored in a freezer when not in use, so that the cooling medium 32 is in a solid state. When the cooling medium 32 melts and/or rises above a predetermined temperature, it may quickly be detached from the system and replaced with a new vessel 26 recently removed from the freezer and containing colder, perhaps still frozen cooling medium 32.

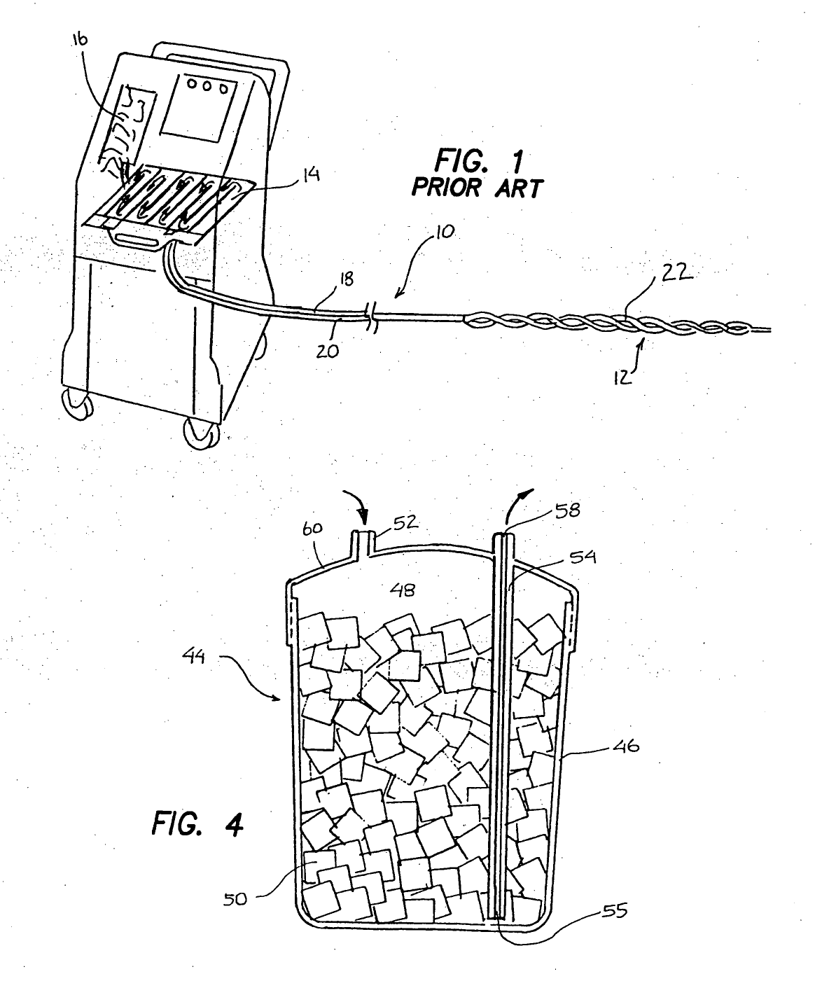

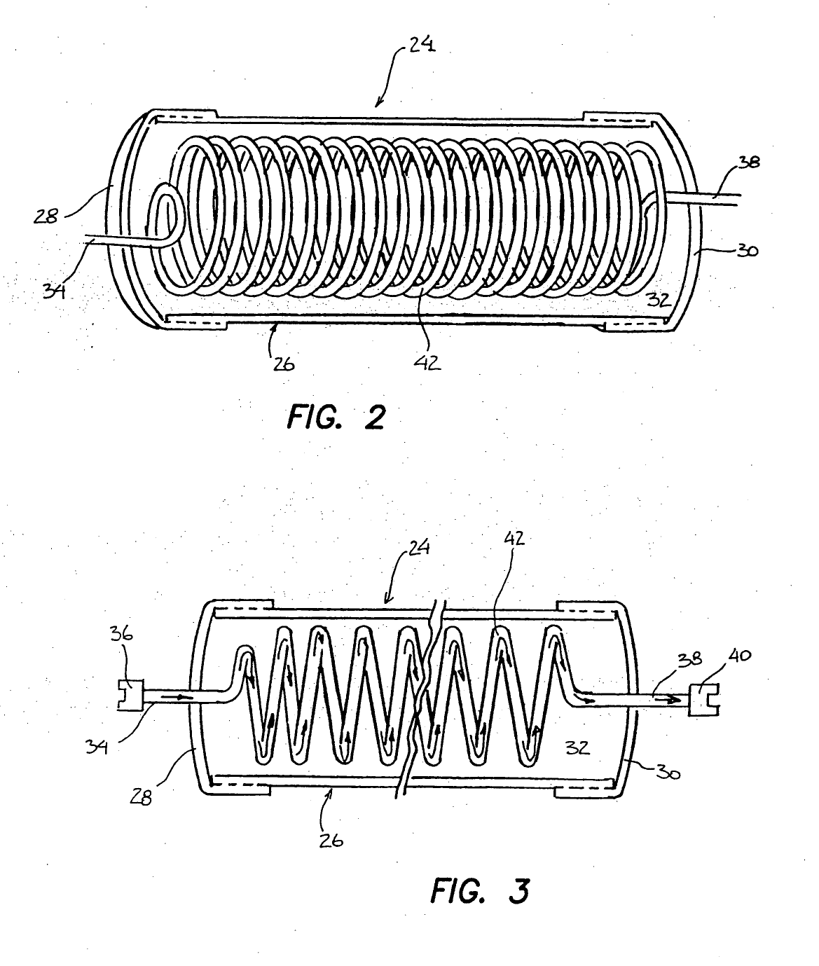

A cooling device 44 according to an alternate embodiment of the invention is shown in FIG. 4. The device 44 comprises a vessel 46, such as for instance, an insulated pressure vessel, defining a cooling chamber 48 containing a cooling medium 50 such as, for instance, a saline ice bath. An inlet tube 52 at the top of the vessel 46 receives heat exchange fluid from a fluid source and introduces the fluid into the cooling chamber 48, where the fluid directly contacts the cooling medium 50. The vessel is preferably configured for vertical orientation, so that the fluid entering through the inlet tube is pulled downward by gravity toward the bottom of the vessel 46. The fluid at the bottom of the vessel 46 then enters the entrance end 56 of a narrow outlet tube 54 and is drawn upward, for instance by capillary action, to exit end 58 of the tube 54, from whence it passes into the inlet lumen of the heat exchange catheter.

The vessel 46 preferably includes a removable cap or closure 60 that allows ice to be added to the chamber 48 when needed. A seal is preferably provided between the cap and the side wall of the vessel 46 to prevent heat loss and/or fluid leakage.

A method of cooling a heat transfer fluid using the device 44 is substantially the same as the method described above in connection with the first embodiment, except that rather than storing the device, filled with cooling medium, in a freezer prior to use, it may be preferable to store the vessel 46 in an empty state at room temperature, and to simply add the mixture of saline and ice just before use. In addition, instead of replacing the vessel 46 with a new vessel when the cooling medium becomes too warm, a user may simply add more ice to reduce the temperature.

While this invention has been described with respect to various specific examples and embodiments, it is to be understood that the invention is not limited thereto and that it can be variously practiced within the scope of the following claims.

1-62. (canceled)

63. A method of cooling a heat transfer fluid for use in a heat exchange catheter having at least one heat transfer surface and at least one flow lumen which permits flow of the heat transfer fluid past the heat transfer surface, the method comprising:
providing a cooling medium in a vessel having an inlet and an outlet and defining an interior chamber;
detachably coupling the inlet of the vessel in fluid communication with a fluid source containing the heat transfer fluid and the outlet of the vessel to the flow lumen of the heat exchange catheter;
passing the heat transfer fluid through the interior chamber to allow heat exchange between the cooling medium and the heat transfer fluid; and
passing the heat transfer fluid from the interior chamber to the flow lumen of the heat exchange catheter.

64. A method according to claim 63, further comprising a step of storing the vessel in a freezer when not in use.

65. A method according to claim 64, wherein the cooling medium has an initial temperature cooler than the heat transfer fluid, and further comprising a step of replacing the vessel with a similar vessel when the temperature of the cooling medium exceeds a predetermined level.

66. A method according to claim 63, wherein the steps of passing the heat transfer fluid through the chamber and passing the heat transfer fluid to the flow lumen of the catheter comprises pumping the heat transfer fluid.

67. A method according to claim 63, further comprising a step of passing the heat transfer fluid through a primary heat exchanger before or after passing the heat transfer fluid through the vessel.

68. A method according to claim 63, wherein the vessel is configured to maintain the heat transfer fluid in a substantially still condition.

69. A method according to claim 63, wherein the fluid comprises saline solution.

70. A method according to claim 63, wherein the method medium comprises saline solution and ice.

71. A method according to claim 63, wherein the method medium comprises a frozen liquid.

72. A method according to claim 63, wherein the method medium comprises a frozen gel.

73. A method according to claim 63, wherein the method device is a direct contact heat exchanger.

74. A method according to claim 63, wherein the vessel is a pressure vessel.

75. A method according to claim 63, wherein the vessel is insulated.

76. A method according to claim 63, wherein the method device includes a removable closure allowing the chamber to be conveniently refilled.

77. A method according to claim 76, further comprising a sealing element between the closure and a wall of the vessel.

78. A method according to claim 63, wherein the outlet is positioned relative the inlet so that flow from the inlet to the outlet is assisted by gravity.

79. A method according to claim 63, wherein the vessel is configured to be positioned vertically, with the inlet opening into an upper portion of the chamber, and wherein the outlet comprises an outlet tube having an entrance end opening into a lower portion of the chamber and an exit end proximate the upper end of the chamber.

80. A method according to claim 79, wherein the outlet tube is sufficiently narrow to allow the fluid to rise in the tube by capillary action.

81. A method according to claim 63, wherein the method device includes a duct communicating with the inlet and outlet to allow the fluid to pass through the chamber, the duct having a side wall separating the fluid from the method medium.

82. A method according to claim 81, wherein the duct comprises a helical coil.

83. A method according to claim 63, further comprising a first quick-disconnect fastener provided at the inlet of the method device and a second quick-disconnect fastener provided at the outlet of the method device for allowing the method device to be quickly connected to and disconnected from the fluid source and the heat exchange catheter.

84. A method according to claim 63, further comprising a step of adding ice to the vessel when the temperature of the cooling method medium exceeds a predetermined level.
